(19) United States
(12) Patent Application Publication
(10) Pub. No.: US 2018/0161604 A1
(43) Pub. Date: Jun. 14, 2018

(54) BOLUS ASSEMBLY AND ULTRASOUND PROBE ASSEMBLY FOR USE WITH AND/OR INCLUDING SAME

(71) Applicant: SONACARE MEDICAL, LLC, Charlotte, NC (US)

(72) Inventors: Rodrigo Chaluisan, Charlotte, NC (US); Ralf Seip, Charlotte, NC (US)

(21) Appl. No.: 15/842,805

(22) Filed: Dec. 14, 2017

Related U.S. Application Data
(60) Provisional application No. 62/433,989, filed on Dec. 14, 2016.

(51) Int. Cl. A61N 7/00

(52) U.S. Cl. CPC A61N 7/00; A61B 8/4281

(57) ABSTRACT

A bolus assembly is configured to receive and be removably coupled to a transducer assembly of an ultrasound probe assembly. The bolus assembly includes a housing assembly and at least one sealing member. The housing assembly has a body having first and second ends, and a sidewall extending therebetween, the body forming a cavity defined by the first end, second end, and sidewall, the cavity being configured to receive at least one transducer of a transducer assembly, a first passage extending through the second end of the body, the sidewall including at least one opening spaced-apart from the passage; and an acoustically transparent and distensible membrane attached to an entire outer periphery of the opening. The sealing member has a through hole and is positioned within the passage. The through hole is configured to receive the shaft. The sealing member is sealingly engaged with the shaft within the passage.

### CROSS-REFERENCE TO RELATED APPLICATION

This application claims priority to U.S. Provisional Application No. 62/433,989, filed Dec. 14, 2016 and titled “Robotic Therapeutic Ultrasound System and Method,” which is herein incorporated by reference.

### FIELD

In one embodiment, the presently disclosed technology relates generally to bolus assemblies, and more particularly to bolus assemblies for therapeutic and/or diagnostic ultrasound probe assemblies.

### BACKGROUND

It is known to use therapeutic ultrasound in a clinical setting for the treatment of a multitude of diseases and conditions in a non-invasive or minimally invasive manner. One such example is described in U.S. Patent Application Publication No. 2014/0243677. In therapeutic ultrasound systems, coupling the ultrasound energy from the ultrasound transducer assembly of the ultrasound probe assembly to the patient is typically accomplished through a water-filled conformal bolus assembly of the ultrasound probe assembly. Specifically, prior art therapeutic probe assemblies are commonly filled with a coupling fluid (i.e., water), and include a distensible membrane that allows the probe assemblies to be conformable. This achieves several benefits. First, the coupling fluid provides an acoustic path for the ultrasound waves to travel from the transducer assembly into the region of interest. Second, the probe assembly conforms to the curved anatomy of the patient to provide good contact over a large area. Third, the fluid and the membrane houses and protects the transducer assembly in a convenient structure, and manages the temperature of the transducer assembly (typically to cool it) and the patient interface (to either cool it to protect it, or to warm it for comfort). Finally, the bolus assembly and coupling fluid may be used to control the position of the transducer assembly with respect to the patient, such as disclosed in United States Patent Application Publication No. 2016/0236013.

In general, ultrasound probe assemblies for ultrasound applications (e.g., imaging/diagnostic, non-destructive testing/evaluation, therapeutics) tend to be application-specific, in order to take advantage of particular geometries, target characteristics and workflow, while minimizing constraints due to size, signal to noise ratio, and other requirements. The phrase “application-specific” means that the probe assembly and/or the bolus assembly are generally only effective for one type or style of medical procedure. Additionally, in many cases, the probe assembly houses mechanical actuators (e.g., motors) that aid with positioning the transducer on the target, and/or manipulate the position and orientation of the transducer so as to be able to deliver therapeutic ultrasound energy to a larger target volume. Mechanical transducer translation within the probe assembly can be further augmented with electronic beam steering to target a larger volume. In these cases, the transducer assembly is tightly linked and coupled to the bolus assembly, which is also tightly configured for a specific implementation. Such an arrangement produces probes that are excellent for a single application, but restricts or even prevents their use for other applications. As a result, employing different probe assemblies for different applications is relatively inefficient, and tends to be rather expensive.

### SUMMARY

In light of the above, there is room for improvement in bolus assemblies and/or ultrasound probe assembly technology. Embodiments of the presently disclosed technology overcome the above and other drawbacks of prior art designs and satisfy the above-outlined and other objectives.

In one aspect of the presently disclosed technology, a bolus assembly is configured to receive and be removably coupled to a transducer assembly of an ultrasound probe assembly. The bolus assembly can include a housing assembly comprising a body having a first end, an opposing second end, and a sidewall extending between the first and second ends. The body can form a cavity defined by the first end, the second end, and the sidewall. The cavity can be configured to receive at least one transducer of a transducer assembly of an ultrasound probe assembly. A first passage can extend through the second end of the body. The sidewall can include at least one opening. The at least one opening can be spaced-apart from the first passage. An acoustically transparent and distensible membrane can be attached to an entire outer periphery of the at least one opening. At least one sealing member can have a through hole. The sealing member can be positioned within the first passage of the housing assembly. The through hole of the sealing member can be configured to receive the shaft of the transducer assembly therethrough. The sealing member can be sealingly engaged with the shaft within the first passage of the housing assembly.

As another aspect of the presently disclosed technology, a bolus assembly is configured to receive and be removably coupled to a transducer assembly of an ultrasound probe assembly. The bolus assembly can include a housing assembly having a body with a first end, an opposing second end, and a first passage extending through the body from the first end to the second end. A cap member can be removably connected to the first end of the body. The cap member can form a cavity configured to receive at least one transducer of a transducer assembly of an ultrasound probe assembly. The cap member can have at least one opening. An acoustically transparent and distensible membrane can be attached to an entire outer periphery of the at least one opening.

As yet another aspect of the presently disclosed technology, an ultrasound probe assembly includes a transducer assembly having a shaft and at least one transducer positioned at a distal end of the shaft. A bolus assembly can receive and being removably coupled to the transducer assembly. The bolus assembly can include a housing assembly comprising a body having a first end, an opposing second end, and a sidewall extending between the first and second ends. The body can form a cavity defined by the first end, the second end, and the sidewall. The cavity can be configured to receive at least one transducer of a transducer assembly of an ultrasound probe assembly. A first passage can extend through the second end of the body. The sidewall can include at least one opening. The at least one opening can be spaced-apart from the first passage. An acoustically transparent and distensible membrane can be attached to an entire outer periphery of the at least one opening. At least one sealing member can have a through hole. The sealing member can be positioned within the first passage of the housing assembly. The through hole of the sealing member can be configured to receive the shaft of the transducer assembly therethrough. The sealing member can be sealingly engaged with the shaft within the first passage of the housing assembly.

### BRIEF DESCRIPTION OF THE DRAWINGS

The foregoing summary, as well as the following detailed description of the presently disclosed technology, will be better understood when read in conjunction with the appended drawings. For the purpose of illustrating the presently disclosed technology, there are shown in the drawings various illustrative embodiments. It should be understood, however, that the presently disclosed technology is not limited to the precise arrangements and instrumentalities shown. In the drawings:

FIG. 1 is a partially exploded perspective view of an ultrasound probe assembly in accordance with one embodiment of the presently disclosed technology;

FIG. 2 is a partially exploded perspective view of an ultrasound probe assembly in accordance with another embodiment of the presently disclosed technology;

FIG. 3 is a schematic view of a portion of an ultrasound probe assembly in accordance with yet another embodiment of the presently disclosed technology;

FIG. 4 is a schematic view of a portion of an ultrasound probe assembly in accordance with still a further embodiment of the presently disclosed technology;

FIG. 5 is a schematic view of a portion of an ultrasound probe assembly in accordance with another embodiment of the presently disclosed technology;

FIG. 6 is a schematic view of a portion of an ultrasound probe assembly in accordance with a further embodiment of the presently disclosed technology;

FIG. 7 is a schematic view of a portion of an ultrasound probe assembly in accordance with another embodiment of the presently disclosed technology;

FIG. 8 is a schematic view of a portion of an ultrasound probe assembly in accordance with yet another embodiment of the presently disclosed technology;

FIG. 9 is a schematic view of a portion of an ultrasound probe assembly in accordance with still another embodiment of the presently disclosed technology;

FIG. 10 is a partially simplified, partially schematic, partially exploded view of the ultrasound probe assembly according to FIG. 9; and

FIG. 11 is a partially exploded perspective view of an ultrasound probe assembly in accordance with an embodiment of the presently disclosed technology.

### DETAILED DESCRIPTION

While systems, apparatus and methods are described herein by way of examples and embodiments, those skilled in the art recognize that the systems, apparatus and methods of the presently disclosed technology are not limited to the embodiments or drawings described. It should be understood that the drawings and description are not intended to be limited to the particular form disclosed. Rather, the presently disclosed technology covers all modifications, equivalents and alternatives falling within the spirit and scope of the appended claims. Any headings used herein are for organizational purposes only and are not meant to limit the scope of the description or the claims.

As used herein, the words “is” and “may” are used in a permissive sense (i.e., meaning having the potential to) rather than the mandatory sense (i.e., meaning must). As employed herein, the term “number” shall mean one or an integer greater than one (i.e., a plurality). As employed herein, the term “coupling member” refers to any suitable connecting or tightening mechanism expressly including, but not limited to, rivets, screws, bolts and the combinations of bolts and nuts (e.g., without limitation, lock nuts), washers and nuts, zip ties, and wire ties. Similarly, the words “include,” “including,” and “includes” mean including, but not limited to. Unless specifically set forth herein, the terms “a,” “an” and “the” are not limited to one element but instead should be read as meaning “at least one.” The terminology includes the words noted above, derivatives thereof and words of similar import.

As described below, various embodiments of the presently disclosed technology may be readily combined or even omitted. While the presently disclosed technology is described with reference to (therapeutic and/or diagnostic) ultrasound or high intensity focused ultrasound (“HIFU”), the presently disclosed technology is not so limited and has applicability to other fields and uses. Furthermore, like elements among different embodiments are distinguished by a magnitude of one hundred (100).

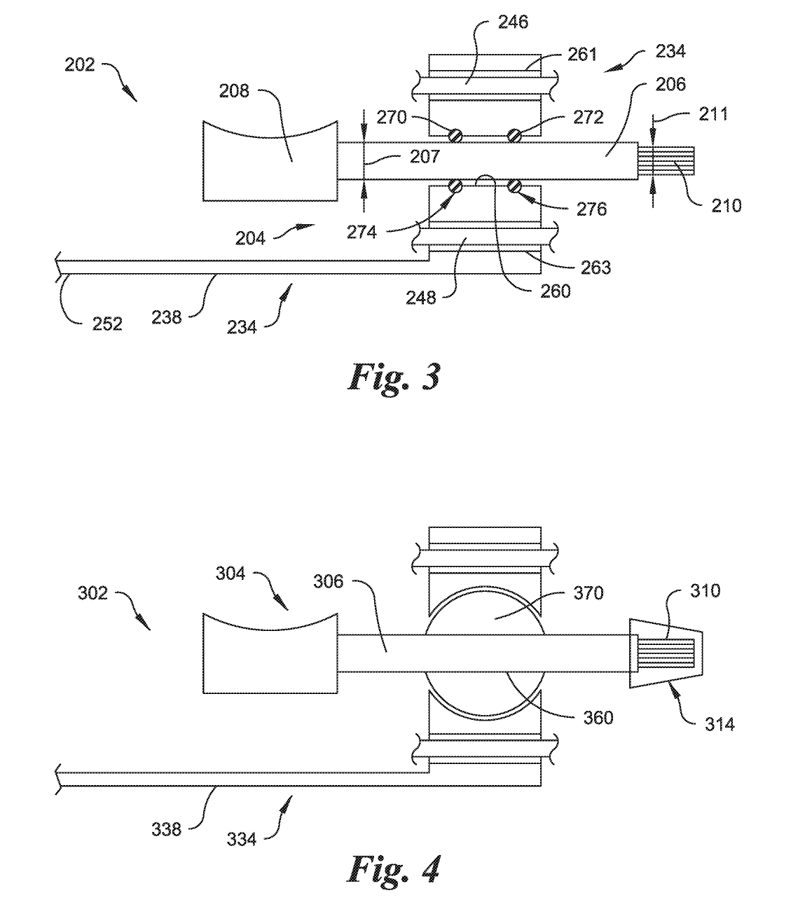

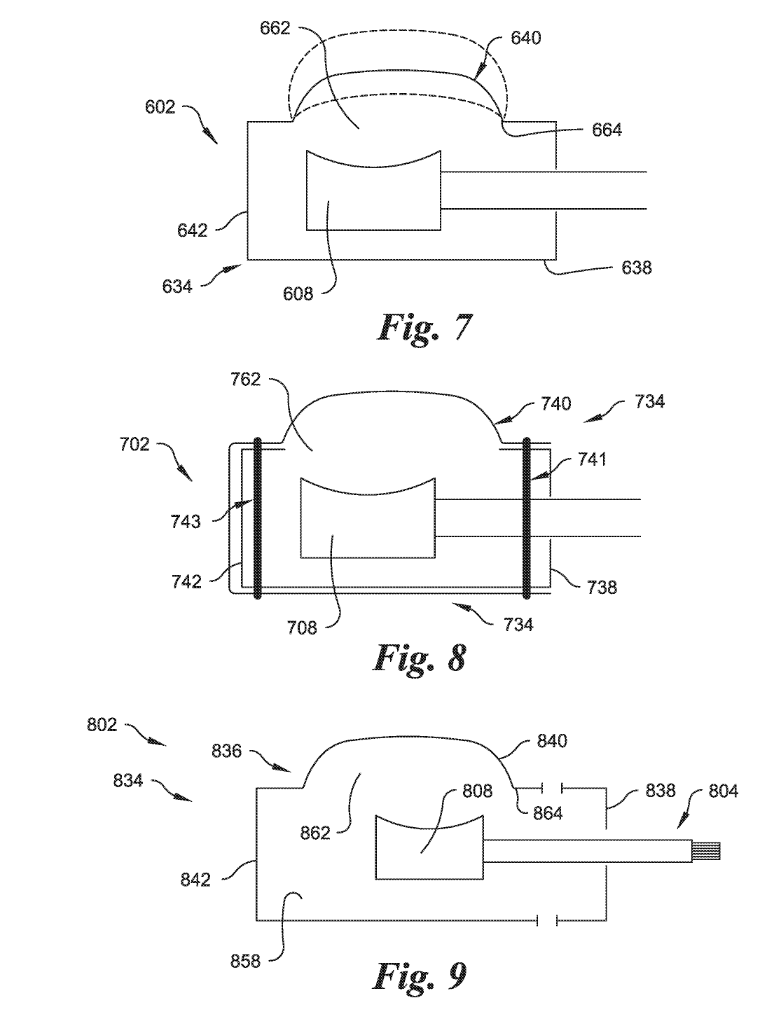

Description of similar or identical features between the embodiments may be omitted herein for the sake of brevity and convenience only. For example, features described or shown in the embodiment that references FIG. 3 can also be included or part of one or more of the embodiments shown in FIGS. 7-10, and vice versa. Thus, any feature of one particular embodiment can form part of another embodiment, unless otherwise noted,

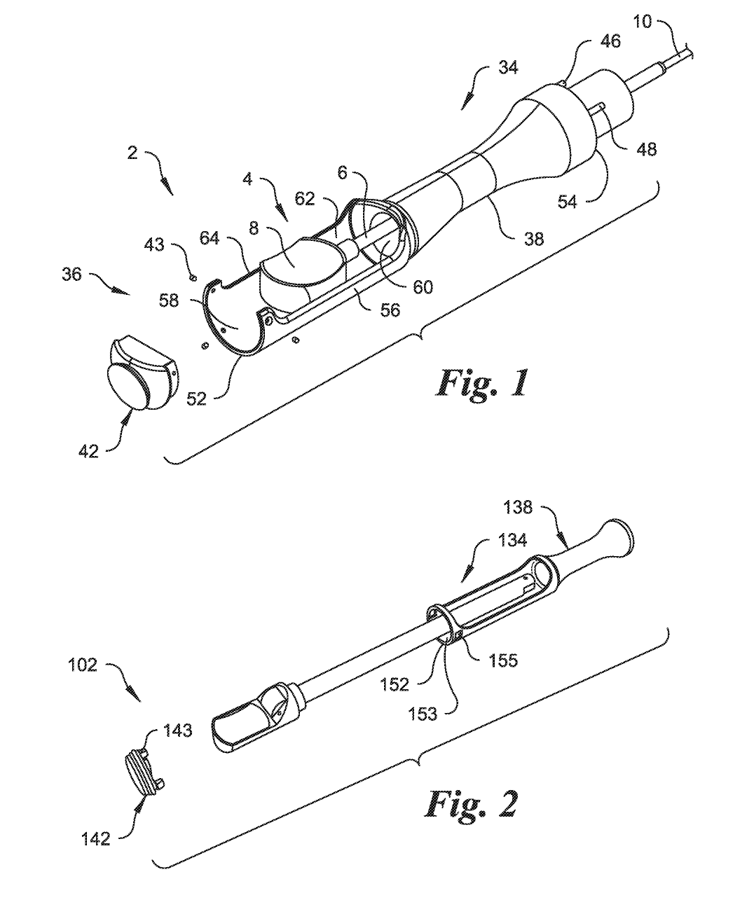

One way to solve the drawback(s) of the prior art is to provide an ultrasound probe assembly in which a transducer assembly of the probe assembly is removably coupled to, but not integrally connected with, a bolus assembly of the probe assembly. In this manner, because transducer assemblies tend to be less application-specific (i.e., more versatile or usable in different medical procedures) than bolus assemblies, users can, in order to provide probe assemblies for different applications, employ disposable bolus assemblies. Bolus assemblies, as compared to transducer assemblies, are relatively inexpensive. As such, once a probe assembly has been used, a user can simply, and relatively easily, remove the transducer assembly from the bolus assembly and dispose of the bolus assembly. Furthermore, the same transducer assembly can relatively easily be employed with many different bolus assemblies because each given bolus assembly can relatively easily be removably coupled to and de-coupled from the transducer assembly. The aforementioned advantages will be discussed in greater detail in connection with the various embodiments shown in FIGS. 1-11.

FIG. 1 shows a partially exploded perspective view of an ultrasound probe assembly 2. The probe assembly 2 includes a transducer assembly 4 and a bolus assembly 34 that can receive and be removably coupled to the transducer assembly 4. The example transducer assembly 4 includes a shaft 6, at least one transducer (e.g., only one example transducer 8 is shown in FIG. 1) positioned at a distal end of the shaft 6, and an interconnect mechanism 10 positioned at or near a proximal end of the shaft 6. The interconnect mechanism 10 is electrically connected with the transducer 8, and is configured so as to be electrically connected with, for example, a mechanical/robotic positioning subsystem (not shown) and/or electrical transducer driving electronics (for imaging, therapy, not shown), in order to transmit energy from the subsystem to the transducer 8. In one embodiment, the interconnect mechanism 10 includes the electrical connections that connect the transducer elements/arrays/crystals to an outside amplifier/controller, together with any of the other electrical connections that typically need to be made (e.g., connect to an embedded EEPROM or device, which can contain probe serial number information, calibration information, etc.). In one example embodiment, the interconnect mechanism 10 is located external with respect to the bolus assembly 34.

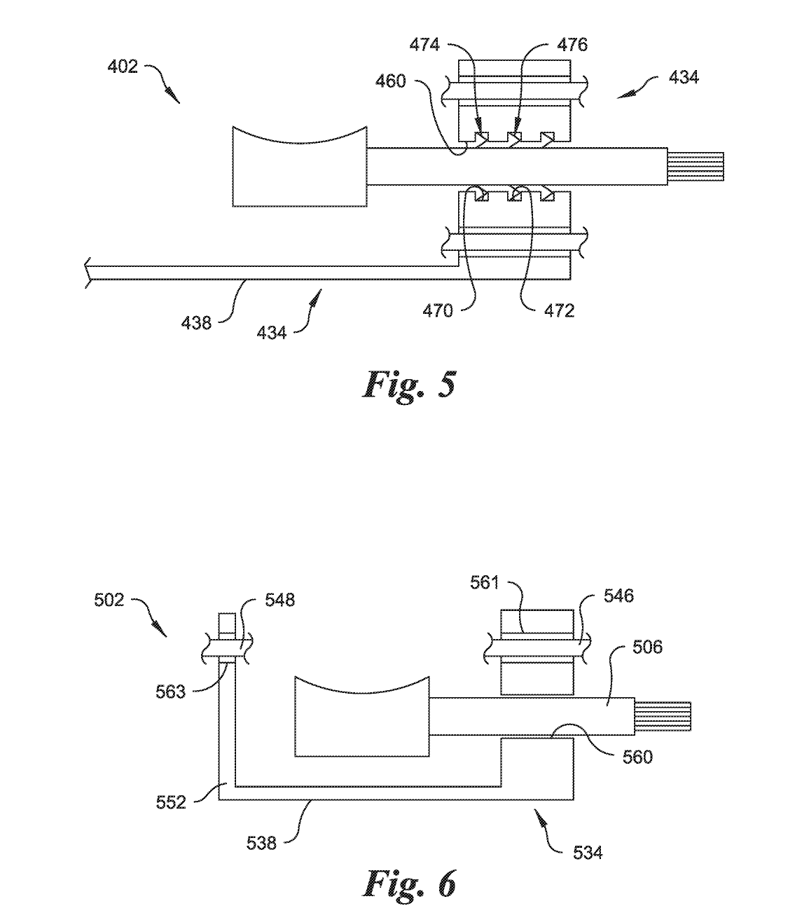

The bolus assembly 34 can include a housing assembly 36 and at least one sealing member (not shown in FIG. 1, but see, for example, sealing members 274, 276, 474, 476 in FIGS. 3 and 5). The housing assembly 36 can include a body 38 and an acoustically transparent and distensible membrane (not shown in FIG. 1, but see, for example, membranes 640, 740, 840 shown in FIGS. 7-9). In one example embodiment, the housing assembly 36 can include a cap member 42 and at least one coupling member or fastener 43. The cap member 42 can include an arcuate top surface and a skirt depending or extending outwardly therefrom. The skirt can have a different or smaller size or shape than a remainder of the cap member 42, so as to facilitate attachment of the cap member 42 with the body 38. The example body 38 has a first or distal end 52, an opposing second or proximal end 54, and a sidewall 56 extending between the first and second ends 52, 54. The body 38 can be sized, shaped and/or configured to form a cavity 58 defined by the first end 52, the second end 54, and the sidewall 56. The cavity 58 can be configured to receive the entire transducer 8 and at least a portion of the shaft 6. Thus, a diameter or cross-sectional dimension of the transducer 8 and the shaft 6 can be less than that of the cavity 58, thereby permitting the transducer 8 and the shaft 6 to move within or with respect to the cavity 58.

In one embodiment, the body 38 can include a number of passages extending through the second end 54 of the body 38. For example, the body 38 can include a first passage 60 for receiving the shaft 6 of the transducer assembly 4, and second and third passages (e.g., not shown in FIG. 1, but see, for example, second and third passages 261, 263 shown in FIG. 3) for allowing fluid to enter and/or exit the cavity 58. The sidewall 56 can include at least one opening 62 that is spaced-apart from the first passage 60.

In order to assemble the probe assembly 2 of one embodiment, the interconnect mechanism 10 and the shaft 6 can be inserted at least partially through the first passage 60 until the transducer 8 is positioned in the cavity 58. Subsequently, the cap member 42 is removably connected to the first end 52 of the body 38. In one embodiment, at least a portion of the first end 52 of the body 38 can surround at least a portion (e.g., the skirt) of the cap member 42, and the coupling members 43 can extend through the first end 52 and at least partially into the cap member 42 in order to connect the cap member 42 to the first end 52 of the body 38. Additionally, the membrane (see, for example, membranes 640, 740, 840 shown in FIGS. 7-9) can be attached to an entire outer periphery 64 of the opening 62 such that the transducer 8 is surrounded by the membrane, the body 38, and the cap member 42.

In one embodiment, the body 38 has at least one fluid inlet and at least one fluid outlet in order to allow fluid to be added into and removed from, respectively, the cavity 58. Specifically, the body 38 can include second and third passages (not shown in FIG. 1, but see second and third passages 261, 263 in FIG. 3) that extend from the cavity 58 through the second end 54 of the body 38. Furthermore, in one example embodiment shown in FIG. 1, the bolus assembly 34 further includes a first conduit 46 and a second conduit 48 each extending at least partially into (or completely through) the second passage and the third passage, respectively. In an alternative embodiment, a distal end of each of the first and second conduits 46, 48 can be in abutting contact with a proximal end of the second and third passages, respectively. Regardless of which of the above-described configurations is employed, this arrangement allows for filling and emptying the bolus assembly 34 with ultrasound coupling fluid (e.g., sterile or non-sterile water), and/or allows for the removal or gas bubbles present or lodged within the inner volume of the bolus assembly 34. This will be discussed in greater below, in connection with the bolus assembly 234 of FIG. 3.

The first and second conduits 46, 48 can be made from respective first and second material, and the body 38 may be made from a third material that is different from the first and second material. The first and second material can be the same, or they can be different. A suitable alternative bolus assembly (not shown) may be configured such that second and third passages function as respective first and second conduits, thereby eliminating the need for the separate first and second conduits 46, 48.

The body 38 and the cap member 42 of the bolus assembly 34 can each also be unitary or integral components made from a single material (e.g., without limitation, molded components made from suitable thermoplastic materials). This can be advantageous in that the bolus assembly 34 is relatively simple and inexpensive to manufacture. As such, as bolus assemblies in accordance with the disclosed concept no longer need to be integrally or permanently connected to the transducer assemblies, the bolus assembly 34 and suitable alternative bolus assemblies can be disposed of after use at relatively minimal expense, and can be manufactured in many different configurations and/or geometries. Therefore, a given or single transducer assembly can be employed with many different bolus assemblies.

The probe assembly 2 of FIG. 1 is shown after a transducer assembly 4 has been inserted into the bolus assembly 34, but before the cap member 42 has been coupled to the body 38, and before the flexible membrane has been attached to the bolus assembly 34. This highlights (i) the acoustic window (i.e., the opening 62 through which the transducer 8 can propagate ultrasound energy), (ii) the position of the transducer 8 within the bolus assembly 34, (iii) the fluid inlet/outlet ports (e.g., the conduits 46, 48), and (iv) the interconnect mechanism 10 positioned at the proximal end of the transducer shaft 6. As assembly of the probe assembly 2 is relatively simple (e.g., inserting at least a portion of the shaft 6 and the interconnect mechanism 10 through the first passage 60, removably coupling the cap member 42 to the first end 52 of the body 38, and sealingly attaching the membrane (not shown in FIG. 1) to the outer periphery 64 of the opening 62), the bolus assembly 34 overcomes many of the aforementioned drawbacks of the prior art. For example, different bolus assemblies in accordance with the disclosed concept that are constructed in a similar manner as the bolus assembly 34 may be employed with the same transducer assembly 4 in order to deliver therapeutic ultrasound energy to other targets of a patient. Furthermore, de-coupling bolus assemblies from transducer assemblies advantageously enables different transducer assemblies, which might otherwise be application-specific, to share a common positioning system (e.g., without limitation, a general-purpose robotic positioner or arm), thereby expanding the application space or usability of the ultrasound system.

In one embodiment, the probe assembly 2 and/or the bolus assembly 34 can include a mechanical interface at its base that allows an external positioning system (e.g., without limitation, a robot arm, an articulated arm, a mechanical holding arm) to attach to the bolus assembly 34 in a keyed, stable/solid and unique manner. Accordingly, the positioning system can be used to manipulate and place the probe assembly 2 onto the area of interest, such that the transducer 8 is able to deliver ultrasound energy to the target tissue. In one embodiment, this mechanical interface is separate from the mechanical interface (e.g., interconnect mechanism 10) between the transducer assembly 4 and external positioning system. The transducer assembly 4 can have a second, separate, but similar, positioning system interface that is keyed, stable/solid, and provides a strong and reliable attachment point between the transducer assembly 4 and the positioning system. As such, when it is attached, the positioning system can manipulate the transducer assembly 4 within the bolus assembly 34 unimpeded, without further displacing or changing the position of the bolus assembly with respect to the patient.

In an extracorporeal application, for example and without limitation, the external positioning system can place the probe assembly 2 at or near the target location of the patient (e.g., skin). The probe assembly can further be attached to the patient via straps, belts and/or held in place by a different/separate positioning system. This allows the positioning system (e.g., robotic positioning system) to disengage from the bolus assembly 34, and attach to the transducer assembly 4 instead or separately via a keyed mechanism on the transducer shaft 6, so as to be able to manipulate the transducer 8 within the bolus assembly 34 under robotic/computer/user control to carry out the steps needed to deliver the ultrasound therapy.

In an intracavity application (e.g., trans-rectal, trans-vaginal), for example and without limitation, the external positioning system can place the probe assembly 2 within the cavity of the patient. In these cases, the bolus assembly 34 is designed in such a way as to stay in place without further attachment to the positioning system (e.g., robotic positioning system), although additional attachments to the patient may still be considered in this application. This can be accomplished by providing a bulbous tip (i.e., proximate the cap member 42), a narrow neck (i.e., proximate the tip of the arrow for 38 shown in FIG. 1), and then again a larger structure on an opposing end (i.e., proximate the second end 54 of the body 38 shown in FIG. 1), so that the entire assembly is held in place via its neck, and is unable to move in or out of the cavity because of the shapes of the ends. After positioning, the positioning system can disengage from the bolus assembly 34, and attach to a proximal end of the transducer assembly 4, and begin to manipulate this assembly (e.g., rotating, translating, angulating), so as to be able to accomplish the requirements of delivering ultrasound energy to the target tissue.

In a laparoscopic application, for example and without limitation, it is possible that the positioning system places the bolus assembly 34 by itself into position first. Then, the user places the transducer assembly 4 into the bolus (i.e., as in these cases the neck of the bolus assembly 34 may not be particularly narrow, as compared to the extracorporeal or intracavity implementations), and seals the structure. Once in place, the bolus assembly 34 can be filled with fluid, the positioning system attached to the shaft 6, and the ultrasound treatment executed.

The bolus assembly 34 can also contain a mechanical or other machine-readable structure at its base (e.g., without limitation, a barcode, a set of dimples or bumps, and/or a key or the like), which identifies the bolus assembly 34 by its type to the system (e.g., a computer system in a separate console) when the positioning system grabs or engages the bolus assembly 34. This identifier allows a therapeutic system console and the positioning system to know what type of bolus assembly has been connected to it automatically, and configure itself by loading in representative parameters specifically used for that specific bolus assembly. Such parameters may include, for example and without limitation: overall size and resulting XYZ travel extents of the transducer assembly 4, and volume (e.g., to guide the filling/emptying of the ultrasound coupling fluid via a fluid management system or loop, and to set upper/lower limits for safe operation and degassing time). In some cases, the bolus identifier may also uniquely specify the intended application, if only one bolus assembly is used for one specific application, for example.

FIG. 2 shows a bolus assembly 134 and an ultrasound probe assembly 102 in accordance with another non-limiting embodiment of the presently disclosed technology. The first end 152 of the body 138 can from a continuous or unitary ring having a central through hole 153, and one or a plurality of spaced-apart side through holes 155. The cap member 142 can include one or a plurality of spaced-apart deflection members 143 extending outwardly from a base of the cap member 142. In operation of one embodiment, the cap member 142 can be configured to extend at least partially through the central through hole 153 and into a cavity of the bolus assembly. In this manner, the deflection members 143 are structured to at least slightly deflect and be positioned at least partially through the side through holes 155 in order to sealingly connect the cap member 142 with the first end 152 of the body 138. As such, in one embodiment, the cap member 142 is removably connected to the first end 142 of the body 138 by a snap-fit mechanism without the use of additional tools. Although the disclosed concept has been described thus far in association the cap members 42, 142 being removably connected to the first ends 52, 152 by way of the coupling members 43 and the snap-fit connection, respectively, it will be appreciated that suitable alternative connection mechanisms are contemplated herein (e.g., without limitation, screw on connections and/or twist-on and lock mechanisms).

When comparing FIG. 1 and FIG. 2, it is evident that that the shaft of different transducer assemblies can have varying thicknesses, widths and/or diameters. For example, the shaft 6 shown in FIG. 1 has a diameter that is smaller less than that of the shaft shown in FIG. 2. The larger, wider and/or thicker shafts can accommodate and/or surround objects, such as one or more cables or fluid passages, used to connect the transducer(s) to one or more separate components, such driving electronics. Thus, the transducer assembly can accommodate fluid inlet and fluid outlet ports, instead of the bolus assembly. The bolus assembly 134 can be sized, shaped and/or configured to accommodate the larger, wider and/or thicker shaft of a particular transducer assembly.

FIG. 3 shows a proximal portion of a bolus assembly 234 and an ultrasound probe assembly 202 in schematic form in accordance with another non-limiting embodiment of the presently disclosed technology. The bolus assembly 234 of FIG. 3 (as well as FIGS. 4 and 5) can include additional structure or features distal to the portion shown. For convenience only, FIG. 3 (and FIGS. 4 and 5) show only a proximal portion of the bolus assembly 234 that interacts with and/or accepts at least a portion of the transducer assembly 204. The transducer 208 can be positioned between the first end 252 of the body 238 and the second and third passages 261, 263 within the cavity. In one embodiment, the second and third passages 261, 263 are each configured to receive a corresponding one of the first and second conduits 246, 248. As discussed above, the first and second conduits 246, 248 and corresponding second and third passages 261, 263 are located so as to facilitate the removal of gas bubbles and/or the injection or removal of fluid. Specifically, the outlet port can be the first conduit 246 and the second passage 261, each positioned at a top portion of the bolus assembly 234. This is also represented with the example bolus assemblies 334, 434 of FIGS. 4 and 5.

For clarity and ease of understanding, FIG. 3 shows a diameter, for example, of each of the second and third passages 261, 263 as being appreciably larger than a diameter, for example, of the first and second conduits 246, 248. While this arrangement is certainly plausible and beneficial in certain applications, the diameters may be closer in size or have an identical size between the second passage 261 and the first conduit 246, as well as between the third passage 263 and the second conduit 248.

In one embodiment, the first passage 260 can include one or a plurality of spaced-apart, annular-shaped grooved regions 270, 272. A sealing member 274, 276, which may be O-rings, is located in and sealingly engaged with a corresponding one of the grooved regions 270, 272. The sealing members 274, 276 can have through holes and are positioned within the first passage 260 in order to receive the shaft 206 of the transducer assembly 204 therein. Additionally, in one embodiment, the sealing members 274, 276 are sealingly engaged with the shaft 206 within the first passage 260. In other words, each sealing member 274, 276 can form a flexible and fluid-tight interface with the shaft 206 such that ultrasound coupling fluid (e.g., water) is generally prevented from flowing through the first passage 260. While the bolus assembly is being shown in association with the two sealing members 274, 276, it is within the scope of the disclosed concept for a suitable alternative bolus assembly to employ any suitable alternative number of sealing members.

Continuing to refer to FIG. 3, in one embodiment, the shaft 206 can have a thickness or first diameter 207, and the interconnect mechanism 210 can have a thickness or second diameter 211 less than that of the shaft 206. This geometry provides advantages for assembly of the probe assembly 202. Specifically, when the shaft 206 and interconnect mechanism 210 are inserted through the first passage 260, the relatively small thickness or diameter 211 of the interconnect mechanism 210 allows the interconnect mechanism 210, which can include sensitive electrical contacts and/or other components, to pass through the first passage 260 without a significant risk of being damaged by first passage 260.

FIG. 4 shows a bolus assembly 334 and an ultrasound probe assembly 302 in accordance with another non-limiting embodiment of the presently disclosed technology. The body 338 of the bolus assembly 334 has a generally spherical-shaped portion 370, which can be movable relative (e.g., rotatable) to a remainder of the body 338. As shown in FIG. 4, the spherical-shaped portion 370 can define, form and/or surround the first passage 360. Alternatively, the spherical-shaped portion 370 can fit within the first passage 360.

In this manner, the spherical-shaped portion 370 advantageously functions as a sealing member that receives and is sealingly engaged with the shaft 306 of the transducer assembly 304. In other words, the spherical-shaped portion 370 is configured so as to form a watertight interface with the shaft 306 in order to prevent ultrasound coupling fluid from flowing in or through the first passage 360 when the shaft 306 is inserted into the bolus assembly 334.

In one embodiment, the transducer assembly 304 includes a guard member 314 (a simplified form of one embodiment is shown in FIG. 4) removably coupled to the interconnect mechanism 310. Optionally, the guard member 314 can be tapered or have a partially conical-shape. When the shaft 306 and interconnect mechanism 310 are inserted through the first passage 360, the guard member 314 provides a mechanism to protect the relatively sensitive electrical components of the interconnect mechanism 310. After the shaft 306 has been properly positioned in the bolus assembly 334 and the interconnect mechanism 310 extends outside of the bolus assembly, the guard member 314 can be removed, thereby exposing the sensitive electrical components of the interconnect mechanism 310 so that they may be connected to an appropriate structure. This connects the transducer assembly of the probe assembly 302 to a console assembly, thereby allowing appropriate control signals and/or energy to be delivered to/from the transducer. Each respective transducer assembly could employ a guard member substantially the same as the guard member 314 in order to protect the respective interconnect mechanisms.

FIG. 5 shows a bolus assembly 434 and an ultrasound probe assembly 402 in accordance with another non-limiting embodiment of the presently disclosed technology. The first passage 460 of the body 438 can include one or a plurality of spaced-apart, annular-shaped grooved regions 470, 472. A sealing member 474,476 can be located in and sealingly engaged with a corresponding one of the grooved regions 470, 472. In the example of FIG. 5, the sealing members 474, 476 are any one of U-cup seals, Teflon seals, and/or spring seals. FIG. 5 only identifies two sets of grooved regions and sealing members with a reference number, but less or additional sets (e.g., three corresponding sets of groove regions and sealing members) are possible. The sealing members 474, 476 provide a fluid-tight interface with the shaft 406 so as to prevent ultrasound coupling fluid from flowing in the first passage 460.

In each of the bolus assemblies 234, 334, 434 shown in FIGS. 3-5, the example structures (e.g., the sealing members 274, 276, the spherical-shaped portion 370, and the sealing members 474, 476) are capable of accepting and sealingly holding the corresponding transducer shafts 206, 306, 406 to the bolus assemblies 234, 334, 434. Furthermore, the sealing members 274, 276, the spherical-shaped portion 370, and the sealing members 474, 476 advantageously allow the transducer assemblies to move in/out of, to easily rotate within, and to move angularly with respect to, the bolus assemblies 234, 334, 434, thereby allowing for relatively easy insertion and removal of the transducer shafts 206, 306, 406, without allowing fluid (e.g., water) to pass therethrough. Furthermore, the interface between the sealing members 274,276, the spherical-shaped portion 370, and the sealing members 474, 476 with the corresponding first passages 260, 360, 460 may be primed with or include a lubricant or other friction-reducing mechanism (e.g., without limitation, surface coating and/or low-friction material) in order to facilitate and enable the transducer assemblies to relatively easily be inserted and removably coupled to the bolus assemblies 234, 334, 434 in a generally fluid-tight manner.

FIG. 6 shows a bolus assembly 534 and an ultrasound probe assembly 502 in accordance with another non-limiting embodiment of the presently disclosed technology. The first conduit 546 (and/or the second passage 561) can extend through the second or proximal end 554 of the bolus assembly 534, as can the shaft 506 and the first passage 560. In contrast to the previous embodiments, the second conduit 548 (and/or the third passage 563) is located at or can extend through the first or distal end 552 of the bolus assembly 534. In one embodiment, the first conduit 546 provides a pathway for fluid to enter the cavity of the body 538, and the second conduit provides a pathway for fluid to exit the cavity of the body 538. As such, it will be appreciated that bolus assemblies in accordance with the disclosed concept may have many different configurations. Furthermore, the instant configuration (i.e., locating the first conduit 546 on one side of the bolus assembly 534 and the second conduit 548 on an opposing side of the bolus assembly 534) advantageously ensures that the circulating ultrasound coupling fluid will relatively easily remove excess heat given off by the transducer during operation.

FIG. 7 shows a bolus assembly 634 and an ultrasound probe assembly 602 in accordance with another non-limiting embodiment of the presently disclosed technology. It will be appreciated that an acoustically transparent and distensible membrane 640 can be attached to the entire outer periphery 664 of the opening 662 of the body 638. The membrane 640 can be attached to interior surface or the exterior surface of the body 638. In one embodiment, the body 638 (optionally including a removable cap member 342) and the membrane 640 surround the transducer 608. In one example embodiment, the membrane 640 may be a latex, silicone, or other similar material that is configured to completely cover the opening 662 and prevent or inhibit the passage of liquid therethrough. Furthermore, the membrane 640, which in the example of FIG. 7 is pre-attached to the body 638, is configured to conform to the shape of the target tissue of the patient. See, for example, dashed lines in FIG. 7 representing different positions the single membrane 640 could conform to, depending on the patient and the pressure applied to the target tissue. Additionally, the dashed lines of FIG. 7 are also indicative of how the membrane 640 might expand or contract, depending on whether ultrasound coupling fluid is added into or removed from, respectively, the cavity of the bolus assembly 634. This advantageously allows the distance between the transducer 608 and the target tissue to be changed, thereby aiding in transducer positioning. Moreover, when the transducer 608 is in place within the bolus assembly 634, and the membrane 640 is secured to the outer periphery 664, ultrasound coupling fluid may be circulated to allow the probe assembly 602 to be prepared for ultrasound therapy application.

FIG. 8 shows a bolus assembly 734 and an ultrasound probe assembly 702 in accordance with another non-limiting embodiment of the presently disclosed technology. The bolus assembly 734 is similar to the bolus assembly 634 of FIG. 7 described above, except that the membrane 740 is configured to be attached by the user. Specifically, the bolus assembly 734 can include a sealing mechanism, such as one or a plurality of spaced-apart O-rings 741,743, configured to sealingly connect the membrane 740 to the body 738 and/or a cap member. That is, the membrane 740 surrounds at least a portion of the body 738 and/or the cap member, and is sealingly connected to the same by the O-rings 741,743. The sealing mechanism is configured to provide sufficient force to hold the membrane 740 in place, so as to prevent fluid from leaving the cavity of the body 748 through the opening 762 enclosed by the membrane 740.

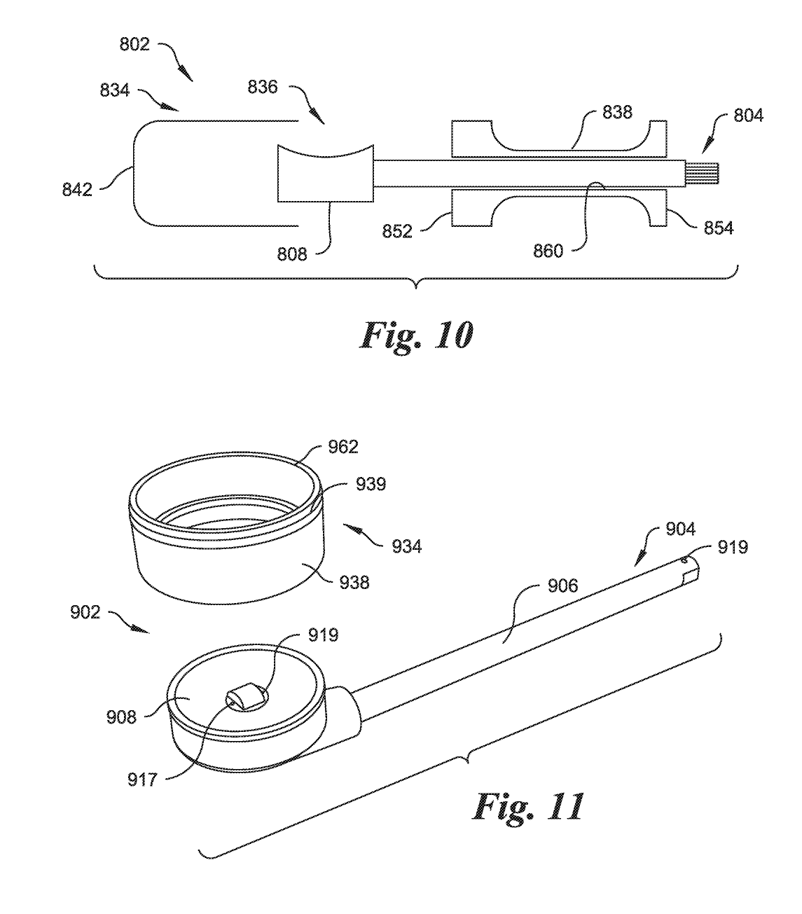

FIGS. 9 and 10 show a bolus assembly 834 and an ultrasound probe assembly 802 in accordance with another non-limiting embodiment of the presently disclosed technology. The bolus assembly 834 is similar to one or more of the bolus assemblies 34, 134, 234, 334, 434, 534, 634, 734 discussed above, with a few notable distinctions. For example, while the housing assembly 836 can include or be formed of the body 838, the membrane 840, and the cap member 842 removably connected to the first end 852 of the body 838, the cavity 858 that receives the transducer 808 of the transducer assembly 804 can be formed by the cap member 842. Furthermore, the opening 862 can be formed in the cap member 842, and the membrane 840 is attached to the entire outer periphery 864 of the opening 862 of the cap member 842. In this embodiment, the cap member 842 is removable from the body 838, as described in one or more of the earlier embodiments. However, in this embodiment, the cap member 842 is generally larger than the body 838.

Referring to FIG. 10, the body 838 can include a first or distal end 852, a second or proximal end 854 opposite the first end 852, and a first passage 860 extending through the body 838 from the first end 852 to the second end 854. The body 838 can have a smaller thickness or diameter in a mid-portion thereof, while the first and second ends 852, 854 can have a larger thickness or diameter. At least a proximal or “open” end of the cap member 842 can contact, engage or sealingly mate with a portion of the body 838, such as the first end 852 thereof. In one example embodiment, the bolus assembly 834 may be configured so as to have any suitable sealing member (e.g., without limitation, sealing members 274, 276, 474, 476 shown in FIGS. 3 and 5) positioned within the first passage 860 of the housing assembly 836.

Similar to one or more of the embodiments described above (e.g., see FIGS. 8 and 9), the cap member 842 can include an opening, spaced-apart from the proximal end thereof, that is enclosed or surrounded by an acoustically transparent and distensible membrane. Also, in a manner similar to one or more of the embodiments described above (e.g., see FIGS. 3-6), the body 838 can include one or more spaced-apart fluid passages. In one embodiment, one of the fluid passages has a fluid inlet at one point on the body 838, and another of the fluid passages has a fluid outlet at a separate point on the body 838. These features have not been shown in these figures or further described herein for the sake of brevity and convenience only.

Continuing to refer to FIGS. 9 and 10, additional advantages are achieved with the bolus assembly 834 of this embodiment. Specifically, many different types or styles of transducers, such as the transducer 808 shown schematically in FIGS. 9 and 10, are measurably or significantly larger than the corresponding shaft of the transducer assembly, thus not allowing bolus assemblies to be placed over the transducer assembly, but rather requiring the transducer assembly to be placed within the bolus assembly. As such, the bolus assembly 834 may be implemented with the two primary components (e.g., the body 838 and the cap member 842) being connected by, for example, a screw-on or clip-on connection, and/or an access hatch. Such an implementation also enables trans-rectal applications, where the requirement to maintain a thin bolus neck (placed in the sphincter) during treatment provides for patient comfort, while still allowing for the use of a bolus tip and transducer that is larger (e.g., thicker) in size than this neck of the bolus assembly, or any other applications where a thinner neck in the body of the bolus assembly is required that would not accommodate the entire transducer, but is able to accommodate the transducer assembly shaft.

In one implementation, the cap member 842 can be a single, unitary component that is user installable, which would relatively easily connect to the body 838. After the attachment of the cap member 842 to the body 838, the bolus assembly 834 would be mechanically complete. In cases where the transducer assembly 804 (i.e., the transducer(s), the shaft, and the interconnect mechanism) is smaller (e.g., thinner) than the neck (e.g., see the tip of the lead line for reference number 838 in FIG. 10) of the bolus assembly 834, the aforementioned bolus assembly 834 may not be preferable.

FIG. 11 shows a bolus assembly 934 and an ultrasound probe assembly 902 in accordance with another non-limiting embodiment of the presently disclosed technology. The bolus assembly 934 can include a body 938 having an opening 962 at a first or upper end thereof and an opening at a second or lower end thereof. Optionally, the body 938 can have a generally circular (e.g., cylindrical) or annular-shape. One or more sealing members (e.g., without limitation, O-ring 939) can be removably or permanently coupled to the body 938, such as at or near the upper end thereof. The O-ring 939 is configured to sealingly attach a flexible, acoustically transparent, and distensible membrane (not shown in FIG. 11) to the body 938 to enclose the opening 962 at the upper end of the body 938. The body 938 can be configured to surround (e.g., be located external with respect to) one or more transducers 908 of the transducer assembly 904, and be sealingly and/or removably connected to the transducer assembly 904.

Similar to previous embodiments, the transducer assembly 904 can include a shaft 906 and the transducer 908 can be positioned at a distal end of the shaft 906. Unlike previous embodiments, the transducer assembly 904 can include or surround a first passage 917 and a second passage 919 spaced-apart therefrom. Each passage 917, 919 can extend through the transducer 908 and through the shaft 906. One of the passages 917, 919 can allow fluid to enter the transducer assembly 904 and the bolus assembly 934, and the other passage 917, 919 can allow fluid to exit the transducer assembly 904 and the bolus assembly 934. In this manner, transducer coupling fluid may enter and exit a cavity formed by the body 938 and the transducer 908, in order to be propagated to target tissue of a patient.

As evidence from the above description and the appended drawings, the bolus assemblies of the presently disclosed technology can be quickly and easily coupled to and de-coupled from corresponding transducer assemblies. In one example embodiment, the bolus assembly is removably coupleable to one or more transducer assemblies without any intermediate parts or components.

A description of one embodiment of how to employ the disclosed concepts will now be provided. A sterile pouch can contain a bolus assembly (e.g., in two parts: the body that accepts the transducer assembly, which may already contain the treatment window and acoustically transparent membrane, and its cap member) and a flexible membrane, a transducer shaft guiding guard member, and additional accessories (e.g., one or more O-rings and/or ultrasound gel), that when assembled, complete the bolus assembly. A user may then open a sterile, or non-sterile, pouch containing a transducer assembly. The user may then attach the guard member to the distal end of the transducer assembly, and feed it though the base of the bolus assembly, so that the transducer is positioned inside the body of the bolus assembly, and interconnect mechanism (and optionally a portion of the shaft) extends outside of the bolus assembly. The cap member may then be connected to the body of the bolus assembly (e.g., without limitation, snapped in, screwed in, etc.), and, if not part of the bolus assembly, the flexible membrane may then be applied to the bolus assembly. The shaft guiding guard member may then be removed.

The completed bolus assembly, which is mated with the transducer assembly, is now attached to the positioning system (e.g., without limitation, a robotic positioning system). This frees up the user's hands to make three remaining connections: (1) attach the fluid inlet conduit to the bolus assembly, (2) attach the fluid outlet conduit to the bolus assembly, and (3) attach the transducer's interconnect mechanism to the console's interconnect. The bolus assembly may now be primed, either automatically or manually, with the coupling fluid. This may be done using, for example, a therapeutic ultrasound coupling fluid management system. The bolus assembly may now de-bubbled, either automatically or manually. After connecting the transducer assembly to the console, priming and debubbling the bolus assembly, the system is ready to be placed on/inside the patient.

Bolus assembly placement can be accomplished manually or with the positioning system, and varies slightly depending on the application (e.g., extracorporeal, intracavity, or laparoscopically). Once properly positioned, the positioning system disengages from the bolus assembly, and is attaches to a proximal end of the shaft of the transducer assembly, thereby being ready for manipulation within the bolus assembly to execute ultrasound treatment. The coupling fluid provides acoustic coupling between the transducer(s) and the target tissue, and is hermetically contained within the bolus assembly due to its sealed structure. At the completion of the treatment, the procedure described above is reversed: the positioning system is detached from the transducer shaft, the connections to the transducer are broken or separated, and the probe assembly is removed from the patient. The fluid may then be drained from the bolus assembly, prior to removing/breaking the acoustic membrane and bolus assembly cap member, for transducer assembly removal. The bolus assembly may be discarded, and the transducer assembly can be cleaned, sterilized, and/or otherwise readied for its next use (or discarded as well), as needed.

The above disclosed systems, apparatuses, methods and description of generic embodiments of the presently disclosed technology are provided to enable any person skilled in the art to make or use the disclosed concept. Various modifications to the embodiments described herein will be readily apparent to those skilled in the art, and the generic principles described herein can be applied to other embodiments without departing from the spirit or scope of the presently disclosed technology. Thus, it is to be understood that the description and drawings presented herein represent a functional generic embodiment of the presently disclosed technology and are, therefore, representative of the subject matter which is broadly contemplated by the presently disclosed technology. It is further understood that the scope of the presently disclosed technology fully encompasses other embodiments that may become obvious to those skilled in the art and that the scope of the presently disclosed technology is accordingly limited by nothing other than the appended claims.

I/We claim:

1. A bolus assembly configured to receive and be removably coupled to a transducer assembly of an ultrasound probe assembly, the bolus assembly comprising:
a housing assembly comprising:
a body having a first end, an opposing second end, and a sidewall extending between the first and second ends, the body forming a cavity defined by the first end, the second end, and the sidewall, the cavity being configured to receive at least one transducer of a transducer assembly of an ultrasound probe assembly, a first passage extending through the second end of the body, the sidewall including at least one opening, the at least one opening being spaced-apart from the first passage;
an acoustically transparent and distensible membrane attached to an entire outer periphery of the at least one opening; and
at least one sealing member having a through hole, the at least one sealing member being positioned within the first passage of the body, the through hole of the sealing member being configured to receive a shaft of the transducer assembly therethrough, the sealing member being sealingly engaged with the shaft within the first passage of the body.

2. The bolus assembly of claim 1, wherein the housing assembly further comprises a cap member removably connected to the first end of the body.

3. The bolus assembly of claim 2, wherein the first end of the body has a through hole, and wherein the cap member is sealingly connected with the first end of the body.

4. The bolus assembly of claim 2, wherein at least a portion of the first end of the body surrounds at least a portion of the cap member, and wherein the housing assembly further comprises at least one coupling member extending through the first end and at least partially into the cap member to connect the cap member to the first end.

5. The bolus assembly of claim 1, wherein the body has a second passage and a third passage both extending from the cavity through the second end of the body.

6. The bolus assembly of claim 5, further comprising a first conduit and a second conduit each extending at least partially into the second passage and the third passage, respectively, wherein the first conduit is made from a first material, wherein the second conduit is made from a second material, and wherein the body is made from a third material different from the first material and the second material.

7. The bolus assembly of claim 5, wherein the first passage comprises a plurality of annular-shaped grooved regions, and wherein the at least one sealing member comprises a plurality of O-rings each disposed in and sealingly engaged with a corresponding one of the grooved regions.

8. The bolus assembly of claim 5, wherein the at least one sealing member comprises a plurality of sealing members each being selected from the group consisting of U-cup seals, Teflon seals, and spring seals.

9. The bolus assembly of claim 5, wherein the body further has a generally spherical-shaped portion defining the first passage, and wherein the spherical-shaped portion is configured to receive and be sealingly engaged with the shaft.

10. The bolus assembly of claim 1, wherein the body is a unitary component made from a single piece of material.

11. A bolus assembly configured to receive and be removably coupled to a transducer assembly of an ultrasound probe assembly, the bolus assembly comprising:
a housing assembly comprising:
a body having a first end, an opposing second end, and a first passage extending through the body from the first end to the second end,
a cap member removably connected to the first end of the body, the cap member forming a cavity configured to receive at least one transducer of a transducer assembly of an ultrasound probe assembly, the cap member having at least one opening, and
an acoustically transparent and distensible membrane being attached to an entire outer periphery of the at least one opening.

12. The bolus assembly of claim 11, further comprising at least one sealing member having a through hole, the at least one sealing member being positioned within the first passage of the body, the through hole of the sealing member being configured to receive a shaft of the transducer assembly, the sealing member being sealingly engaged with the shaft within the first passage of the body.

13. An ultrasound probe assembly comprising:
a transducer assembly comprising a shaft and at least one transducer positioned at a distal end of the shaft; and
a bolus assembly receiving and being removably coupled to the transducer assembly, the bolus assembly comprising:
a housing assembly comprising:
a body having a first end, an opposing second end, and a sidewall extending between the first and second ends, the body forming a cavity defined by the first end, the second end, and the sidewall, the cavity being configured to receive the at least one transducer, a first passage extending through the second end of the body, the sidewall including at least one opening, the at least one opening being spaced-apart from the first passage;
an acoustically transparent and distensible membrane being attached to an entire outer periphery of the at least one opening; and
at least one sealing member having a through hole, the at least one sealing member being positioned within the first passage of the body, the through hole of the sealing member receiving the shaft of the transducer assembly therethrough, the sealing member being sealingly engaged with the shaft within the first passage of the body.

14. The ultrasound probe assembly of claim 13, wherein the transducer assembly further comprises an interconnect mechanism positioned at or near a proximal end of the shaft, wherein the interconnect mechanism is electrically connected with the at least one transducer, wherein the shaft has a first diameter, and wherein the interconnect mechanism has a second diameter less than the first diameter.

15. The ultrasound probe assembly of claim 14, wherein the transducer assembly further comprises a guard member removably coupled to the interconnect mechanism.

16. The ultrasound probe assembly of claim 13, wherein the body further has a second passage and a third passage both extending from the cavity through the second end of the body.

17. The ultrasound probe assembly of claim 16, wherein the first passage comprises a plurality of annular-shaped grooved regions, wherein the at least one sealing member comprises a plurality of O-rings each disposed in and sealingly engaged with a corresponding one of the grooved regions, and wherein each of the plurality of O-rings is sealingly engaged with the shaft of the transducer assembly.

18. The ultrasound probe assembly of claim 16, wherein the at least one sealing member comprises a plurality of sealing members each being selected from the group consisting of U-cup seals, Teflon seals, and spring seals.

19. The ultrasound probe assembly of claim 16, wherein the body further has a generally spherical-shaped portion defining the first passage, and wherein the spherical-shaped portion is configured to receive and be sealingly engaged with the shaft of the transducer assembly.

20. The ultrasound probe assembly of claim 13, wherein the housing assembly further comprises a cap member connected to the first end of the body, and wherein the at least one transducer is surrounded by the membrane, the cap member, and the body.

21. An ultrasound probe assembly comprising:
a transducer assembly comprising a shaft and at least one transducer positioned at a distal end of the shaft, a first passage and a second passage extending through the shaft and the at least one transducer; and
a bolus assembly being removably coupled to the transducer assembly, the bolus assembly having a body forming a cylinder, and upper end of the body having an opening, a lower end of the body having an opening, the lower end of the body being configured to removably attach to the transducer assembly, an acoustically transparent and distensible membrane being attached to the upper end of the body and surrounding the opening of the upper end of the body.

22. The ultrasound probe assembly of claim 21, further comprising an O-ring surrounding at least a portion of the membrane and the upper end of the body to attach the membrane to the body, and wherein fluid enters the ultrasound probe assembly through one of the first and second passages, and wherein fluid exits the ultrasound probe assembly through the other of the first and second passages.
